(19) United States
(12) Patent Application Publication
(10) Pub. No.: US 2025/0325590 A1
(43) Pub. Date: Oct. 23, 2025

(54) METHOD OF TREATING AN INDIVIDUAL WITH A HEALTH CONDITION WITH FECAL MICROBIOTA TRANSPLANT

(71) Applicant: Sabine Hazan, Ventura, CA (US)

(72) Inventor: Sabine Hazan, Ventura, CA (US)

(21) Appl. No.: 17/831,349

(22) PCT Filed: Feb. 11, 2021
(86) PCT No.: PCT/US2021/017671

§ 371 Date: Jun. 2, 2022

Related U.S. Application Data
(60) Provisional application No. 62/991,190, filed on Mar. 18, 2020.
(60) Provisional application No. 62/991,146, filed on Mar. 18, 2020.
(60) Provisional application No. 62/991,699, filed on Mar. 19, 2020.
(60) Provisional application No. 63/002,486, filed on Mar. 31, 2020.

(51) Int. Cl. A61K 35/24; A61B 1/31; A61B 10/00; A61K 38/14; C12Q 1/689

(52) U.S. Cl. CPC A61K 35/24; A61B 10/0038; A61K 38/14; C12Q 1/689; A61B 1/31; C12Q 2600/156

(57) ABSTRACT

A method of treating an individual with a health condition comprising the steps of: a) screening the individual to determine a level of severity of the health condition; b) acquiring a fecal sample from the individual; c) processing the fecal sample; d) sequencing the fecal sample to determine the individuals microbiome; e) analyzing the sequenced fecal sample, wherein the step of analyzing the sequenced fecal sample comprises: i) comparing the individual's microbiome to the microbiome of a parent of individual; ii) comparing the individual's microbiome to the microbiome of a sibling of the individual; iii) comparing the individual's microbiome to the microbiome of another individual with same health condition; and iv) comparing the individual's microbiome with a health condition to the microbiome of the individual before they acquired the health condition; f) administering vancomycin to the individual; g) performing the fecal microbiota transplant; and h) monitoring the individual.

### CROSS-REFERENCE TO RELATED APPLICATIONS

This application claims priority to and is a national phase entry of International Application No. PCT/US2021/017671, titled “Method of Treating an Individual with a Health Condition with Fecal Microbiota Transplant,” filed Feb. 11, 2021, which claims priority to U.S. Provisional Patent Application No. 63/002,486, titled “Method of Analyzing the Microbiome of Individual Stool Samples,” filed Mar. 31, 2020, U.S. Provisional Patent Application Ser. No. 62/911,699, titled “Autologous Gastrointestinal Microbiota Preservation,” filed Mar. 19, 2020, U.S. Provisional Application Ser. No. 62/991,146, titled “Autologous and Familial Fecal Microbiota Transplant,” filed Mar. 18, 2020, U.S. Provisional Patent Application No. 62/991,190, titled “Method of Analyzing the Microbiome of Individual Stool Samples,” filed Mar. 18, 2020, and U.S. Provisional Patent Application Ser. No. 62/933,891, titled “Method of Treating Disease with Fecal Microbiota Transplant,” filed Nov. 11, 2019, the contents of which are incorporated by reference in their entirety.

### BACKGROUND

The human gastrointestinal (GI) microbiome is a complex, interconnected web of microbes, living in a symbiotic relationship with their host. There are greater than ten times more bacteria in the human body than there are human cells, all in a delicate and ever-changing balance to maintain a healthy GI tract. When this balance is disrupted, a condition known as dysbiosis, or disease, can occur. Traditional methods of treating disease and infection include the use of prescription medications, which come with potentially serious side effects and other issues.

Thus, there is a significant unmet need for assisting individuals suffering from a health condition (including disease and or infection) to achieve with symptomatic relief, and possibly remission, without the use of prescription medications.

### SUMMARY

The present invention addresses this need. The present invention is directed to my method of treating an individual with a health condition with fecal microbiota transplant. The method comprises the steps of: a) screening the individual to determine a level of severity of the health condition; b) acquiring a fecal sample from the individual; c) processing the fecal sample from the individual; d) sequencing the fecal sample from the individual to determine the individuals microbiome; e) analyzing the sequenced fecal sample; f) administering vancomycin to the individual; f) performing the fecal microbiota transplant; and g) monitoring the individual.

The step of acquiring the fecal sample comprises use of a stool sample collection kit or colonoscopy. The stool sample kit comprises: a) at least one stool sample collection vial; b) at least one toilet accessory or seat cover; c) at least one specimen bag; d) at least one pair of gloves; e) an authorization form; f) a patient information card; g) a questionnaire; and h) stool sample collection instructions.

The step of analyzing the sequenced fecal sample comprises one or more of the following: a) comparing the individual's microbiome to the microbiome of a parent of individual; b) comparing the individual's microbiome to the microbiome of a sibling of the individual; c) comparing the individual's microbiome to the microbiome of another individual with same health condition; and d) comparing the individual's microbiome with a health condition to the microbiome of the individual before they acquired the health condition.

The step of administering the vancomycin can comprise administering 250 mg of liquid vancomycin to the individual every 8 hours for 10 consecutive days.

Optionally, the step of administering the vancomycin comprises administering 250 mg of liquid vancomycin to the individual every 8 hours for 6 consecutive weeks.

A stool sample used for the fecal microbiota transplant is donated from one or more of the following: the individual, a genetic family member of the individual, or a third party unrelated to the individual.

### DRAWINGS

These and other features, aspects and advantages of the present invention will be better understood with reference to the following description, appended claims, and accompanying drawings where:

FIG. 1 is a flow chart of a method of treating disease with fecal microbiota transplant, having features of the present invention;

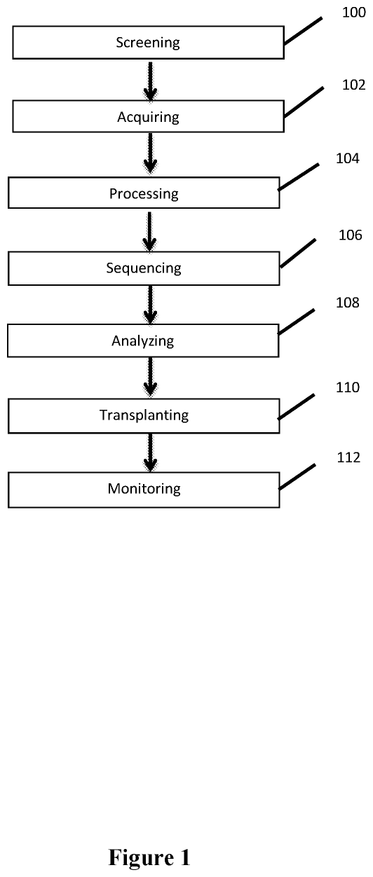

FIG. 2 is a top plan view of a stool collection kit having features of the present invention; and

FIG. 3 is top plan view of the stool collection kit of FIG. 2, wherein the contents have been removed from the box.

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

### Definitions

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers, ingredients or steps.

Crohn's Disease, which is characterized by patchy, transmural inflammation that can affect any part of the gastrointestinal tract, can be defined in two ways. The first is by location: terminal ileal, colonic, ileocolonic, and upper gastrointestinal. The second is by pattern of disease: inflammatory, structuring, or fistulizing, with strictures being the most common complication. Strictures occur because swelling and scar tissue on the intestinal wall results in thickening of these walls which thus narrow the passage. Another complication, fistulas, occurs when there are sores or ulcerations which invade into the surrounding tissues such as bladder, vagina, or skin, causing tunnels between the two areas which should not be present. These fistulas require treatment up to and including surgery, as they often become infected.

Up to 80% of patients with CD will require surgery to remove the inflamed portion of the intestine, with many of the patients requiring additional surgery. Nutritional issues are common in CD, including deficiencies of proteins, calories, and vitamins. These can be caused by inadequate intake, malabsorption, and intestinal loss of protein. Other complications include skin problems, arthritis, osteoporosis, kidney stones and gallstones, inflammation in the eyes or mouth, and other disease of the liver and biliary system. While some of these can resolve with CD treatment, others may require separate treatment.

In the United States it is estimated that 700,000 people suffer from CD, with 1.4 million people worldwide. This number is steadily growing at a rate of 1% per year. With these numbers, CD is now considered to be the second most common chronic inflammatory disorder, after rheumatoid arthritis. While the medical costs associated with CD are high, the total economic burden is much higher due to factors such as lost productivity.

The human gastrointestinal (GI) microbiome is a complex, interconnected web of microbes, living in a symbiotic relationship with their host. There are greater than ten times more bacteria in in the human body than there are human cells in the human body, all in a delicate and ever-changing balance to maintain a healthy GI tract. When this balance is disrupted, a condition known as dysbiosis, or disease, can occur. There is still a debate over whether dysbiosis is a cause of disease or a symptom of it. Naturally, since the microbiome has such a profound impact on human health, including helping humans digest food, make vitamins, and teach human immune cells to recognize pathogens, it plays a vital role in maintaining health. By manipulating the microbiome of patients with disease or infection-induced dysbiosis, the patient's microbiome can be restored to a pre-infection state.

The present invention accomplishes this restoration by utilizing fecal microbiota transplants. The transplants can autologous, meaning utilizing the patient's own stool, collected and stored prior to infection, familial, meaning utilizing a family members stool, or third party donor, meaning the stool is collected from a screened, matched donor that is unrelated to the patient.

The method of the present invention can be used to treat a plurality of diseases, including but not limited to skin cancer, Autism, Clostridioides Difficile Infection, Obesity, Alzheimer's Disease, Crohn's Disease, Myalgic Encephalomyelitis/Chronic Fatigue Syndrome (ME/CFS), Psoriasis, Chronic Urinary Tract Infections, Ulcerative Colitis, Multiple Sclerosis, Chronic Constipation, Lyme Disease, Celiac Disease, Parkinson's Disease, Elevated Cholesterol, Colorectal Cancer, Amyotrophic Lateral Sclerosis (ALS), Fatty Liver, Rheumatoid Arthritis, Anxiety, Obsessive-Compulsive Disorder, Bipolar Disorder, Migraine Headaches, Depression, Diabetes Mellitus, Lupus, Epidermolysis Bullosa, Metastatic Mesothelioma, Eczema, Acne, Irritable Bowel Syndrome, Myasthenia Gravis, and Autism Spectrum Disorders.

In general, the method of the present invention comprises the following steps: screening the individual/patient 100, acquiring a fecal sample from the individual/patient 102, processing the fecal sample from the patient 104, sequencing the fecal sample from the patient 106, analyzing the sequenced fecal sample 108, performing the fecal microbiota transplant 110, and monitoring the patient 112. However, the steps of the method of the present invention can vary depending on whether the transplant is autologous, familial, or third party.

### Autologous Fecal Transplant

The autologous fecal transplant method comprises three main: screening the patient 100, acquiring a sample from the patient 102, and transplanting the patient's own fecal microbiota into the patient 110.

During the step of screening 100, the patient undergoes the following: signing of the consent form, providing their medical history and demographics, having an EKG performed, having their vital signs taken/read, providing their height and weight, and providing the staff with a list of their prior and concomitant medications. Concomitant medications include any form of antibiotics, probiotics, or opiates.

The doctor or staff overseeing the procedure verifies all inclusion and no exclusion criteria are met.

With respect to Crohn's disease specifically, the inclusion criteria comprise the following:

An informed consent form signed by the patient demonstrating that the patient understands the procedures required for the study and the purpose of the study.

The patient is either male or female and between 18 and 75 years of age.

The patient has a probable diagnosis of Crohn's Disease with a Crohn's Disease Activity Index (CDAI) score greater than or equal to 220 and less than or equal to 450 at screening.

The patient's Leukocyte count must be greater than or equal to 3.5×10⁹ at screening.

The patient must have stable cardiac, renal, pulmonary, and hepatic function.

The patient may continue to take medications currently prescribed; however, the patient must be able to discontinue antibiotics prior to fecal microbiota transplant.

The patient must agree to discontinue use of outside probiotics; however, consumption of active culture yogurt is permissible.

The patient must agree to utilize either a barrier contraception method with spermicide or an IUD (intra-uterine device) for the duration of the study.

The exclusion criteria comprise the following:

The patient refuses to sign the informed consent form.

The patient has Crohn's Disease that is isolated to the mouth, upper GI tract, or anus.

The patient has a history of total colectomy with ileorectal anastomosis or

proctocolectomy.

The patient has fistulating Crohn's disease.

The patient has a postoperative stoma, ostomy, or ileoanal pouch.

The patient has short bowel syndrome.

The patient is scheduled for a bowel resection.

The patient has had a bowel perforation within six months of screening.

The patient has known symptomatic obstructive strictures.

The patient was exposed to oral or parenteral antibiotics in the four weeks prior to screening, with the exception of topical antibiotics, which are permitted.

The patient has a positive serology for Hepatitis B, Hepatitis C, or HIV.

The patient is currently diagnosed with, or has a history of, uveitis diagnosed by an optometrist or an ophthalmologist.

The patient has a history of malignancy in the last five years, excluding basal cell carcinoma of the skin or carcinoma in situ of the cervix that has been treated with no evidence of recurrence.

The patient has undergone treatment with total parenteral nutrition.

The patient has a history of active tuberculosis requiring treatment in the past three years.

The patient has a history of drug or alcohol abuse within the past three years.

The patient is a female who is pregnant, intends to become pregnant, or is lactating. This is due to unknown fetal or child effects.

The patient has any clinically significant evidence of any disease not including Crohn's Disease that could interfere with the patient's ability to enter the study.

The patient has an inability to adequately communicate with the investigator or their respective designee and/or comply with the requirements of the entire study.

The patient participated in any experimental drug protocol within the past twelve weeks.

The patient has clinically significant abnormalities in hematology or biochemistry as confirmed by repeat testing based on investigator's discretion.

Once inclusion is confirmed, the staff also confirm the patient has Crohn's Disease based on the following: the patient's clinical history, a colonoscopy, any biopsies taken of the digestive tract, the patient's most recent episode, and a +/− capsule endoscopy. A capsule endoscopy is a procedure that uses a tiny wireless camera to take pictures of the digestive tract.

Bloodwork is also performed on the patient, which includes the following: a blood mycobacterial culture, which is a test to look for the bacteria that cause tuberculosis and other infections caused by similar bacteria, a complete blood count, a chemistry panel, and a C-reactive protein test. C-reactive protein (CPR) is a blood test marker for inflammation in the body. CRP is produced in the liver and its level is measured by testing the blood. CRP is classified as an acute phase reactant, which means that its levels will rise in response to inflammation.

The patient undergoes a urinalysis, a fecal calprotectin test, and uveitis screening.

Fecal calprotectin is a biochemical measurement of the protein calprotectin in the stool. Elevated fecal calprotectin indicates the migration of neutrophils to the intestinal mucosa, which occurs during intestinal inflammation, including inflammation caused by inflammatory bowel disease.

Uveitis is a form of eye inflammation. It affects the middle layer of tissue in the eye wall (uvea). Uveitis warning signs often come on suddenly and get worse quickly. Warning signs include eye redness, pain and blurred vision. The condition can affect one or both eyes.

Next, the patients Crohn's Disease Activity Index (CDAI) is determined. The CDAI is utilized in clinical trials to assess disease activity in Crohn's. CDAI scores can range from 0 to ˜600. A cut-off value of <150 is selected so that most patients below this threshold would be rated by physicians as ‘very well’. The delineator between active and very severe disease is defined as a cut-off value of 450 points. CDAI scores of 150-219 are labelled as mildly active disease and scores of 220-450 as moderately active disease.

Once all the above has been performed by the doctor/provided by the patient, the doctor provides the patient with at least one stool collection kits and instructs the patient on use of the stool collection kits.

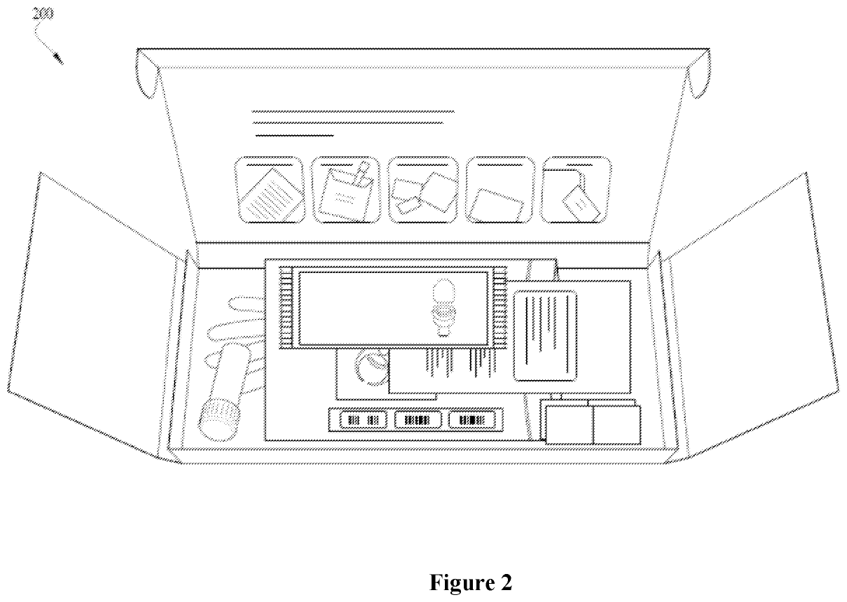

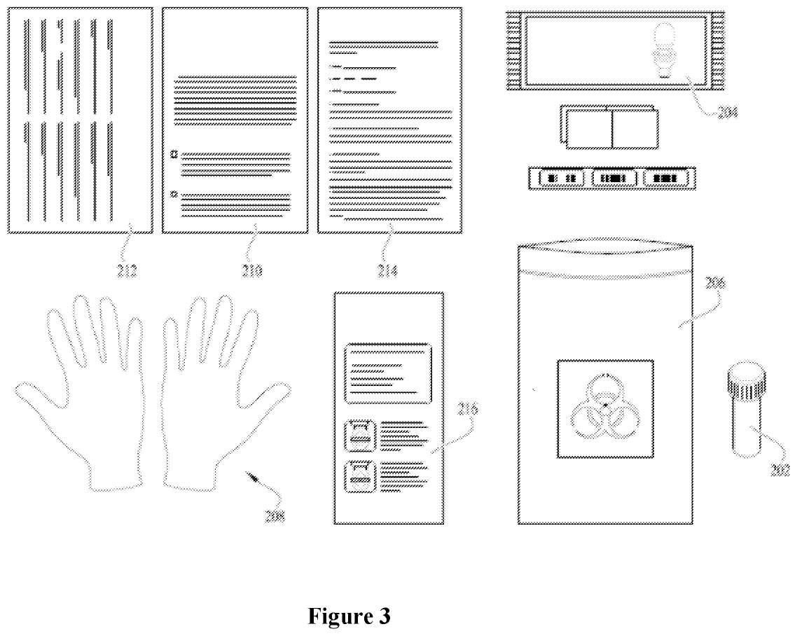

During the step of acquiring a sample 102, regardless of the disease from which the patient is suffering, the following takes place: The step of acquiring a stool sample 102 can either involve the stool sample collection kit 200 or a colonoscopy. The stool sample collection kit 200 is shown in FIGS. 2 and 3 and comprises: at least one stool sample collection vial 202, optionally the vial 200 contains a spoon, at least one toilet accessory or seat cover 204, at least one specimen bag 206, at least one pair of gloves 208, an authorization form 210, a patient information card 212, a questionnaire 214, and stool sample collection instructions 216.

The toilet accessory 204 is in the form of a circular strip of paper that slips over the toilet seat and creates a raised platform on which to provide the voided stool sample.

The stool sample collection instructions 216 are as follows: (1) Correctly position the toilet accessory (i.e. toilet cover) over the toilet seat and put on disposable latex gloves. (2) Unscrew the collection tube cap and use the spoon to scoop one spoonful of the stool sample from the feces. Do not pass the stool sample into the toilet or directly into the collection vial, and do not mix urine or water with the stool sample. (3) Place the stool sample into the collection vial. (4) Tighten the cap and shake to mix the contents thoroughly (and/or invert 10 times) to create a suspension. Some fecal material may be difficult to re-suspend. As long as the stool sample is suspended, the sample is stabilized. Foaming/frothing during shaking is normal. (5) Place the collection vial in the bag labeled “Specimen Bag-Biohazard” and seal the bag. (6) Place the bag back in the collection kit box. (7) Remove toilet cover and let it fall into the toilet bowl. Flush both the toilet cover and excess stool down the toilet. (8) Remove and dispose of gloves. Thoroughly wash hands.

The sample is then processed 104, the microbiome of the sample is sequenced 106 and analyzed 108, and the sample is stored for future use.

In summary, the stool is processed 104 via the following steps:

First, a stool is collected from the patient via an anaerobic method as noted above so that the stool is not exposed to air.

Second, the stool is processed anaerobically in a blender in an anaerobic chamber with normal saline.

Third, the container that the stool is in is kept sealed and stored in a freezer at minus 80 degrees F. until needed for a transplant.

Preferably, the stool sample is stored in a facility much like a sperm bank where individuals can donate their individual stool samples for storage. Most preferably, although not always possible, the individual donates a stool sample (healthy baseline microbiome sample) at birth or at an early stage in life. Optionally, the baseline sample is provided later in life, but pre-hospitalization or pre-epidemic, when the individual is in a healthy state. The purpose is to bank a sample of the individual's healthy microbiome in the event the individual becomes unhealthy and requires a fecal transplant utilizing the healthy sample to reestablish a healthy microbiome. Re-establishing a healthy microbiome will assist the individual in overcoming the current disease, infection, or epidemic.

A more detailed discussion of the processing 104 and sequencing 106 steps are as follows. For these two steps, the following equipment is utilized: centrifuges, pipettes, thermocycler, fluorometers, vortexers, refrigerators/freezers, and a sequencing system (for example, an Illumina NextSeq 550 Sequencing System).

The step of processing the sample 104 includes extracting and purifying patient DNA from the sample. Individual patient DNA is extracted and purified with a DNA extraction kit. Optionally, the QIAmp® PowerFecal® Pro DNA Kit can be used. The DNA extraction kit isolates both microbial and host genomic DNA from stool and gut samples.

In summary, for the DNA extraction step, the stool samples are added to a bead beating tube for rapid and thorough homogenization. Cell lysis occurs by mechanical and chemical methods. Total genomic DNA is captured on a silica membrane in a spin-column format. DNA is then washed and eluted from the membrane and ready for NGS, PCR and other downstream application.

Once the DNA has been extracted, the DNA is then quantitated using a fluorometer. The fluorometer can be a dual-channel fluorometer for nucleic acid quantitation. It provides highly sensitive fluorescent detection when quantifying nucleic acids and proteins.

The following steps are performed when quantitating the sample:

Mix 1-20 microliters of the extracted DNA sample and 200 microliters of dye in a 0.5 ml PCR tube. Mix well by pipetting or vortexing.

The fluorescence is then measured and the nucleic acid concentration is calculated and/or displayed.

Next, the library is prepared. The assay of the present invention is designed to detect all bacteria, viruses, and fungi that reside in the microbiome of the stool samples being evaluated. The assay utilizes an enzymatic reaction to fragment the DNA and to add adapter sequences. Library fabrication includes tagmentation, tagmentation clean-up, and an amplification step followed by another clean-up prior to pooling and sequencing.

The following definitions and abbreviations are used in this section:
BLT: Bead-Linked Transposomes
DNA: Deoxyribonucleic Acid
EPM: Enhanced PCR Mix
EtOH: Ethanol
NGS: Next Generation Sequencing
NTC: No Template Control
PCR: Polymerase Chain Reaction
RSB: Resuspension Buffer
SPB: Sample Purification Beads
TB1: Tagmentation Buffer
TSB: Tagment Stop Buffer
TWB: Tagment Wash Buffer

First, the BLT and TB1 are brought up to room temperature. Then, the BLT and TB 1 are vortexed to mix.

Next, the appropriate volume of DNA is added to each well so that the total input amount is 100 nanograms. The optimal input for this assay is 100 nanograms, however, less DNA input can be utilized.

Next, the appropriate volume of nuclease-free water is added to the DNA samples to bring the total volume to 30 microliters.

Then, the BLT is vortexed vigorously for 10 seconds. Next, 11 microliters of BLT and 11 microliters of TB1 are combined for each sample, creating a tagmentation mastermix. Overage is included in this volume.

Next, the tagmentation master mix is vortexed and the volume is equally divided into an 8-tube strip.

Next, 20 microliters of the tagmentation master mix is transferred to each well containing a sample.

Then, the plate is sealed with Microseal ‘B’ and placed on a thermo cycler preprogrammed with the TAG program. The thermo cycler has a heated lid at 100° C. and reaction volume set to 50 microliters.

Next, the TAG program is run as shown in Table 1:

TABLE 1

| Cycle Step | Temperature | Time |
|---|---|---|
| Step 1 | 55° C. | 15 minutes |
| Step 2 | 10° C. | ∞ |

Once the TAG program is complete, the plate is removed from the thermo cycler.

Next, the Microseal ‘B’ seal is removed and 10 microliters of TSB is added to each sample.

Next, the plate is sealed with a Microseal ‘B’ and placed on the thermo cycler preprogrammed with the PTC program. The thermo cycler has a heated lid at 100° C.

Next, the PTC program is shown in Table 2:

TABLE 2

| Cycle Step | Temperature | Time |
|---|---|---|
| Step 1 | 37° C. | 15 minutes |
| Step 2 | 10° C. | ∞ |

When the PTC program is complete, the plate is removed from the thermo cycler and placed on a magnetic stand. The plate is left on the magnetic stand for about 3 minutes (as long as it takes for the solution to clear).

Once the solution is clear, the Microseal ‘B’ is removed from the plate and the supernatant is removed and discarded.

Next, the plate is removed from the magnetic stand and about 100 microliters of TWB is added. The sample should be pipetted slowly until the beads are fully re-suspended.

Next, the plate is placed back on the magnetic stand and approximately 3 more minutes pass while the solution clears again.

Once the solution clears, the supernatant is removed and discarded.

Next, the plate is removed from the magnetic stand and about 100 microliters of TWB is added. The sample should be pipetted slowly until the beads are fully re-suspended.

Next, the plate is again placed on the magnetic stand for an additional 3 minutes while the solution clears.

Next, 22 microliters of EPM and 22 microliters of nuclease-free water are combined with each sample to form a PCR mastermix. Overage is included in this volume. The PCR mastermix is vortexed and centrifuged.

With the plate on the magnetic stand, the supernatant is removed and discarded.

Next, the plate is removed from the magnetic stand and 40 microliters of PCR mastermix are immediately added directly onto the beads in each sample well.

The mastermix is immediately pipetted until the beads are fully re-suspended. Alternatively, the plate is sealed and a plate shaker is used at 1600 rpm for 1 minute.

Next, the plate is sealed with a Microseal ‘B’ and centrifuged at 280×g for 3 seconds.

Next, 10 microliters of index adapters are added to each sample in the plate. The plate is then centrifuged at 280×g for 30 seconds.

Next, the plate is placed on the thermo cycler that is preprogrammed with the BLT PCR program (and with lid preheated at 100° C.).

The BLT PCR Program is run as shown in Table 3:

TABLE 3

| Cycle Step | Number of Cycles | Temperature | Time | |
|---|---|---|---|---|
| Step 1 | 1 | 68° C. | 3 | minutes |
| Step 2 | 1 | 98° C. | 3 | minutes |
| Step 3 | 5 | 98° C. | 45 | seconds |
| | | 62° C. | 30 | seconds |
| | | 68° C. | 2 | minutes |
| Step 4 | 1 | 68° C. | 1 | minute |

| | | | |
|---|---|---|---|
| Step 5 | 1 | 10° C. | ∞ |

When BLT PCR program is complete, the plate is removed from the thermo cycler and centrifuged at 280×g for 1 minute.

Next, the plate is placed on the magnetic stand and it takes about 5 minutes for the solution to clear.

Next, about 45 microliters of supernatant are transferred from each well of the PCR plate to the corresponding well of a new midi plate.

Then, the midi plate is vortexed and the SPB is inverted multiple times to re-suspend.

Next, about 40 microliters of nuclease-free water is added to each sample well containing supernatant.

Next, about 45 microliters of SPB is added to each sample well. Each sample well is then mixed.

The plate is then sealed and incubated for 5 minutes at room temperature.

Next, the plate is placed on the magnetic stand and it takes about 5 minutes for the solution to clear.

Next, the SPB is vortexed thoroughly and 15 microliters of SPB is added to each well of a new midi plate.

Then, 125 microliters of supernatant is transferred from each well of the first plate into the corresponding well of the second midi plate containing 15 microliters SPB.

Each well of the second midi plate is then mixed and the first midi plate can be discarded.

The second midi plate is sealed and incubated for 5 minutes at room temperature.

The second midi plate is placed on the magnetic stand and it takes about 5 minutes for the solution to clear.

Next, without disturbing the beads, the supernatant is removed and discarded.

While the midi plate is still on the magnetic stand, 200 microliters of fresh 80% EtOH are added to the plate, without mixing. The plate is then incubated for 30 seconds.

Next, without disturbing the beads, the supernatant is removed and discarded.

While the second midi plate is still on the magnetic stand, about 200 microliters of fresh 80% EtOH are added, without mixing. The plate is then incubated for 30 seconds.

Next, without disturbing the beads, the supernatant is removed and discarded. Any residual EtOH is also removed and the second midi plate is allowed to air dry on the magnetic stand for about 5 minutes.

The second midi plate is removed from the magnetic stand and about 32 microliters of RSB is added to the beads.

The second midi plate is then re-suspended and incubated for about 2 minutes at room temperature.

The second midi plate is placed back on the magnetic stand it takes about 2 minutes for the solution to clear.

Once the solution clears, about 30 microliters of supernatant are transferred to a new 96-well PCR plate.

Next, the library is pooled and sequenced.

The following definitions and abbreviations are used in this section:
DNA: Deoxyribonucleic Acid
EtOH: Ethanol
HT1: Hybridization Buffer
NGS: Next Generation Sequencing
NTC: No Template Control
RSB: Resuspension Buffer
SAV: Sequencing Analysis Viewer

The following steps are taken to sequence the DNA 106:
1. Prepare the reagent cartridge for use.
2. Denature and dilute sample libraries.
3. Load pooled sample DNA libraries into the prepared reagent cartridge.
4. Set up and start the DNA sequencing using the selected DNA sequencing machine. The sequencing run can take approximately 27-30 hours to complete.

The bioinformatics pipeline utilizes a computational tool that profiles the microbial communities from metagenomic sequencing data with species level resolution. Patient microbiome profiles are analyzed to ascertain not only the profile of microbes in patient samples but also to identify specific strains, and provide accurate estimation of organismal abundance relative to the overall diversity

Once the DNA is sequenced, the microbiome the individual patient is analyzed 108. The step of analyzing 108 the microbiome of the individual can include the following: comparing the microbiome of the individual to the microbiome of the individual's mother, comparing the microbiome of the individual to the microbiome of a sibling of the individual, comparing the microbiome of the individual with a health condition to the microbiome of another individual with same health condition, and comparing the microbiome of the individual with a health condition to the microbiome of the individual before they acquired the health condition (otherwise referred to as baseline versus non-baseline).

If the individual's baseline microbiome is being used in the analysis step 108, then the above recited steps of acquiring a stool sample 102, processing the stool sample 104, and sequencing the microbiome of the individual 106 are performed at least twice-once before the individual acquires a health condition (known as a baseline) and at least once after the individual acquired the health condition. This is necessary so that the baseline microbiome can be compared to the microbiome when the individual is suffering from a health condition.

Optionally, the steps of acquiring a stool sample 102, processing the stool sample 104, and sequencing the microbiome of the individual 106 are performed for a third time, after the individual has overcome the health condition, to confirm that the individual is healthy again.

During the step of transplanting fecal microbiota 110, the following takes place: When the patient suspects they are out of remission, from whatever disease they are suffering from, they return to the doctor's office for the following: vital signs are taken/read, an EKG is performed, their height and weight is recorded, they provide the doctor with an updated list of prior and concomitant medications, and their bloodwork is also repeated. In the event the patient is suffering from Crohn's disease, the bloodwork includes a blood mycobacterial culture, a complete blood count, a chemistry panel, and a C-reactive protein test. A urinalysis, a fecal calprotectin test, and a uveitis screening are also performed.

Next, if applicable, the patient's Crohn's Disease Activity Index (CDAI) is determined. If their CDAI is greater than 150, the patient will be considered to be out of remission. If their CDAI is greater than 220, the patient will be considered to have moderate to severe disease. If their CDAI is greater than 300, the patient will be considered to have severe disease.

If the patient is determined to be out of remission, an autologous fecal microbiota transplant is scheduled. The patient is also provided with colonoscopy preparation instructions and a bowel cleanse prescription.

Optionally, prior to performing the transplant 110, the patient receives a vancomycin treatment. The vancomycin treatment can be for a period of 10 days or up to 6-weeks, and any length of time in between. The dose of vancomycin is 250 mg of liquid suspension (formulated in a concentration of 500 mg/6 mL) administered orally every 8 hours.

When the patient returns for the transplant, the patient arrives at the surgical center prepped and fasted. The patient signs the informed consent form, and the fecal microbiota transplant is conducted.

The fecal microbiota transplant 110 comprises the following steps:

First, the stool material from the blender is thawed and placed in syringes anaerobically.

Second, the patient is brought into a surgical suite and sedated in order to perform a colonoscopy.

Third, the patient is placed in the Trendelenburg position, where the body is laid supine, or flat on their back on a 15-30 degree incline with their feet elevated above their head. Fourth, 300-400 cc of fecal material is injected into the cecum.

Fifth, the patient is given atropine or diphenoxylate and loperamide for one week to slow the colon so that the stool material will remain inside the digestive tract.

And finally, if a colonoscopy cannot be performed then the blended stool material is administered to the patient via a nasogastric tube or placed in capsules that patient swallows and are then dissolved in cecum.

Once the transplant 110 is completed, the patient is then monitored 112. The step of monitoring 112 involves monitoring the patient for a short period of time before being discharged and returning to the doctor for at least two follow-up visits. The first follow-up visit is typically 28 days after the transplant.

During the first follow-up visit, the patient undergoes a physical examination, their height and weight are recorded, their vital signs are taken/read, an EKG is performed, and their CDAI is determined. Additionally, the patient provides the staff with an updated list of prior and concomitant medications, a stool sample is collected for microbiome analysis, and fecal calprotectin is tested.

The second follow-up visit is typically 28 days after the first follow-up visit. During the second follow-up visit the patient undergoes a physical examination, their height and weight are recorded, and the doctor reviews both the microbiome results and the fecal calprotectin result with the patient. The patient provides the doctor with an updated list of prior and concomitant medications, and the patient's CDAI is also determined (if applicable). If the microbiome results and CDAI score are satisfactory, stool will be collected for future autologous fecal microbiota transplant to be done every 6 months.

### Familial Fecal Transplant

In the event the transplant is a familial transplant, the family member is the stool donor, the individual supplying the stool sample administered to the patient during the fecal microbiota transplant. As such, the family member that is the stool donor undergoes the screening steps 100 outlined above to ensure the family member does not suffer from the disease or infection, and the acquiring a sample steps 102 outlined above.

Preferably, the family member also undergoes the processing 104, sequencing 106, and analyzing 108 steps outlined above to ensure the familial member is a good match for the patient.

Once the familial stool sample has been collected, the patient receiving the sample then undergoes the transplant 110 and monitoring steps 112 outlined above.

### Third Party Donor Fecal Transplant

In the event the transplant is a third party donor transplant, the third party is stool donor, the individual that is supplying the stool sample administered to the patient during the fecal microbiota transplant. As such, the third party undergoes both the screening 100 and the acquiring 102 steps outlined above.

Preferably, the third party donor also undergoes the processing 104, sequencing 106, and analyzing 108 steps outlined above to ensure the third party donor is a good match for the patient.

Once the third party donor stool sample has been collected, the patient receiving the sample then undergoes the transplant 110 and monitoring 112 steps outlined above.

The above methods of treating disease with fecal microbiota transplant can also be used on individuals suffering from Eczema, UTI (urinary tract infection), ME/CFS (mylagic encephalomyelitis/chronic fatigue syndrome), Psoriasis, Skin Cancer, Gout, Acne, IBS (irritable bowl syndrome), elevated cholesterol, Parkinson's disease, Multiple Sclerosis, Ulcerative Colitis, Chronic constipation, celiac disease, Lyme disease, obesity, C. diff, obsessive-compulsive disorder, ALS (amyotrophic lateral sclerosis), metastatic mesothelioma, lupus, epidermolysis bullosa, diabetes mellitus, depression, migraine headaches, bipolar disorder, anxiety, rheumatoid arthritis, fatty liver, and colorectal cancer.

Results from Transplant Studies:

It was determined the Crohn's Disease is multifactorial.

If the transplant is familial, then the stool sample utilized during the transplant comes from a donor that is not the patient themselves, but rather a genetically related family member. With respect to the third party donor, the third party donor is not genetically related to the patient.

In both instances, either the family member or the third party donor donates and stores the sample per the protocol described above. When the patient requires a FMT, the appropriate donor sample is selected and administered to the patient. This is considered a matched donor as their microbiome sequences have been “matched.” To achieve successful GR, a detailed patient analysis is performed, as well as meticulous, precise donor selection. There must be a specific match between patient and donor.

Preferably, the donor/third party is genetically related to the patient.

An individual person's microbiome is an accumulation of bacterial DNA, viral DNA, and fungal DNA. The types, quantity and balance of microbes in a person's microbiome are unique to that individual and can affect their susceptibility or resistance to a variety of health issues. The personal microbiome of an individual is as unique to an individual as a fingerprint.

Finding donors is the most critical, and complex, aspect of treatment, and the most important aspect is to precisely match the donor.

Every human being has a unique microbiome in their gut. This is why finding an appropriate donor is extremely complicated and critical to healing. Donor match is the most important factor because if it is not precise, “the same thing that cures a disease can also cause disease.”

### EXAMPLES

### Example 1: Vancomycin and Familial Fecal Microbiota Transplant for the Treatment of Subjects with Crohn's Disease

Objectives: Improve the core features of Crohn's disease, including the short and long-term effects of the outcomes; Assess gastrointestinal microbiome relative abundance before and after familial fecal microbiota transplant using whole genome shotgun sequencing; Assess safety and tolerability of familial fecal microbiota transplant in subjects with Crohn's disease.

Procedure: Patients with Crohn's disease are treated with vancomycin followed by familial fecal microbiota transplant (FFMT). The vancomycin is prescribed at the second baseline visit and is given for 10 days. For FFMT, the donor provides a fresh stool sample the morning of the procedure. The sample is brought by the donor to the study site for processing. Processing includes emulsification with sterile normal saline at a 1:1 ratio to create a fecal slurry.

Following the FFMT by colonoscopy the caregiver is taught how to prepare and administer the FFMT enemas at home (during the week 4 visit). Table 4 documents the treatment protocol.

TABLE 4

| Intervention | | |
|---|---|---|
| Name | Vancomycin | Familial Fecal Microbiota Trasplant |
| Dose | Suspension of 83 mg/mL | Emulsification |
| Formulation | (500 mg/6 mL) | |
| Route of | Oral | Delivered to the colon directly by |
| Administration | | colonoscope in the first transplant, |
| | | and by enema in all subsequent |
| | | transplants |

Statistical Analysis: The statistical evaluation is performed by an outside statistician using SAS® version 9.3 or higher (Statistical Analysis System, SAS Inc., Cary, NC) software package. Descriptive summaries are given by treatment group and/or overall. The number of subjects within each treatment group of the analysis set is given in each table. Categorical variables are summarized with counts (n) and percentages (%), together with the number of non-missing values. Fisher's exact test for categorical variables is used. The number of non-missing values is used as the denominator for the calculation of percentages. Incidence of adverse events is based on the number of subjects in the respective analysis set and treatment group. Descriptive statistics for continuous variables are comprised of the number of non-missing observations (n), mean, standard deviation (SD), median, minimum (Min) and maximum (Max), if not otherwise stated. Student's t-test and Mann-Whitney for continuous parametric and nonparametric variables are used respectively. ANOVA is used for comparing means between individual groups. When applicable, these summaries are provided by visit. In case of premature withdrawal from the trial, efficacy and safety assessments performed at the time point of withdrawal, are summarized, separately to the planned visits. A P-value<0.05 is taken as significant for all analyses.

Protocol-Required Safety Laboratory Assessment is documented in Table 5.

TABLE 5

| Laboratory | | | |
|---|---|---|---|
| Assessments | Parameters | | |
| Hematology | Platelet Count | RBC Indices: | White blood cell (WBC) |
| | Red blood cell (RBC) | MCV | count with Differential: |
| | Count | MCH | Neutrophils |
| | Hemoglobin | % Reticulocytes | Lymphocytes |
| | Hematocrit | | Monocytes |

Eosinophils
Basophils

| Clinical | Blood urea nitrogen | Potassium | Aspartate | Total and direct |
|---|---|---|---|---|
| Chemistry* | (BUN) | | Aminotransferase | bilirubin |
| | | | (AST)/Serum | |
| | | | Glutamic-Oxaloacetic | |
| | | | Transaminase (SGOT) | |

| Creatine | Sodium | Alanine | Total Protein |
|---|---|---|---|
| | | Aminotransferase | |
| | | (ALT)/Serum | |
| | | Glutamic-Pyruvic | |
| | | Transaminase (SGPT) | |
| Glucose (fasting) | Calcium | Alkaline phosphatase | |

| | |
|---|---|
| Other | Microbiome analysis of stool samples, infectious agent screening of blood |
| Screening | and stool samples of donor and recipient (See Section 4.1, Paragraph |
| Tests | Donor Screening) |

NOTES:
*if INR measured which may indicate severe liver injury (possible Hy's Law), must be reported as an SAE

### Example 2: Vancomycin and Familial Fecal Microbiota Transplant for the Treatment of Subjects with Alzheimer's Disease

Objectives: Improve the core features of Alzheimer's Disease and the short and long-term effects of outcomes; Improve the quality of life of subjects and their caregivers; Assess gastrointestinal microbiome relative abundance before and after familial fecal microbiota transplant using whole genome shotgun sequencing

Procedure: Participants in the study are required to meet the clinician on fifteen occasions, two appointments for screening and baseline assessment, as well as to receive prescription for vancomycin, one appointment for familial fecal microbiota transplant (FFMT) by colonoscopy and twelve appointments to study sites for the post-trial assessment.

Participants in the study are required to meet the clinician on fifteen occasions, two appointments for screening and baseline assessment, as well as to receive prescription for vancomycin, one appointment for familial fecal microbiota transplant (FFMT) by colonoscopy and twelve appointments to study sites for the post-trial assessment.

Familial Fecal Microbiota Transplantation by Colonoscopy: After the baseline data have been collected and the inclusion and exclusion criteria verified, the patient will be scheduled for familial fecal microbiota transplantation (FFMT) at an outpatient surgical center. This transplant will utilize donor stool from a first degree relative (sibling or child)

The patient will prepare for the procedure the day prior by drinking a prescribed regimen of bowel cleanse solution and water. The patent will present for the procedure having fasted and prepped as instructed by the investigator. The patient will undergo the FFMT procedure under anesthesia and be driven home afterwards. Under no circumstances is the patient to drive themselves home.

Familial Fecal Microbiota Transplant by Enema: After 4 weeks the patient will begin FFMT enemas at home utilizing fresh stool from the same donor as the first FFMT. The patient will lie in the lateral decubitus position and the enema will be inserted into the anus. The fecal material will be slowly expelled into the patient's rectum. The patient will remain in the lateral decubitus position for approximately 30 minutes before getting up. The patient may then use the restroom.

Post Treatment Assessment: After FFMT by colonoscopy the patient will be called for post treatment follow-up visits monthly for the Alzheimer's disease assessments MMSE and Qol AD. The patient will also bring a fresh stool sample for microbiome analysis to these appointments. At the 3-month, 6-month, 9-month, and 12-month follow-up visits the patient will be assessed by following parameters: physical examination, vital signs, Adverse Events, concomitant medications, MMSE and Qol AD. The patient will have blood drawn for laboratory analysis and will bring a fresh stool sample for microbiome analysis. Table 6 documents the treatment protocol.

TABLE 6

| Intervention | | |
|---|---|---|
| Name | Vancomycin | Familial Fecal Microbiota Transplant |
| Dose | Suspension of 83 mg/mL | Emulsification |
| Formulation | (500 mg/6 mL) | |
| Route of | Oral | Delivered to the colon directly by |
| Administration | | colonoscope in the first trasplant, |
| | | and by enema in all subsequent |
| | | transplants |

Processing of Sample/Dosing: We will treat patients with AD using vancomycin followed by familial fecal microbiota transplant. The vancomycin will be prescribed at the second baseline visit and will be given for 10 days. This is an open-label study, so blinding is not a component. For FFMT the donor provides a fresh stool sample the morning of the procedure. The sample is brought by the donor to the study site for processing. Processing includes emulsification with sterile normal saline at a 1:1 ratio to create a fecal slurry. Following the FFMT by colonoscopy the caregiver is taught how to prepare and administer the FFMT enemas at home (during the week 4 visit).

Statistical Analysis: The statistical evaluation is performed by an outside statistician using SAS® version 9.3 or higher (Statistical Analysis System, SAS Inc., Cary, NC) software package. Descriptive summaries are given by treatment group and/or overall. The number of subjects within each treatment group of the analysis set is given in each table. Categorical variables are summarized with counts (n) and percentages (%), together with the number of non-missing values. Fisher's exact test for categorical variables is used. The number of non-missing values is used as the denominator for the calculation of percentages. Incidence of adverse events is based on the number of subjects in the respective analysis set and treatment group. Descriptive statistics for continuous variables are comprised of the number of non-missing observations (n), mean, standard deviation (SD), median, minimum (Min) and maximum (Max), if not otherwise stated. Student's t-test and Mann-Whitney for continuous parametric and nonparametric variables are used respectively. ANOVA is used for comparing means between individual groups. When applicable, these summaries are provided by visit. In case of premature withdrawal from the trial, efficacy and safety assessments performed at the time point of withdrawal, are summarized, separately to the planned visits. A P-value<0.05 is taken as significant for all analyses.

Protocol-Required Safety Laboratory Assessment is documented in Table 7.

TABLE 7

| Laboratory | | | |
|---|---|---|---|
| Assessments | Parameters | | |
| Hematology | Platelet Count | RBC Indices: | White blood cell (WBC) |
| | Red blood cell (RBC) Count | MCV | count with Differential: |
| | Hemoglobin | MCH | Neutrophils |
| | Hematocrit | % Reticulocytes | Lymphocytes |

Monocytes
Eosinophils
Basophils

| Clinical | Blood urea nitrogen | Potassium | Aspartate | Total and direct |
|---|---|---|---|---|
| Chemistry* | (BUN) | | Aminotransferase | bilirubin |
| | | | (AST)/ Serum | |
| | | | Glutamic-Oxaloacetic | |
| | | | Transaminase (SGOT) | |

| Creatine | Sodium | Alanine | Total Protein |
|---|---|---|---|
| | | Aminotransferase | |
| | | (ALT)/ Serum | |
| | | Glutamic-Pyruvic | |
| | | Transaminase (SGPT) | |
| Glucose (fasting) | Calcium | Alkaline phosphatase | |

| | |
|---|---|
| Other | Microbiome analysis of stool samples, infectious agent screening of blood and |
| Screening | stool samples of donor and recipient (See Section 4.1, Paragraph Donor |
| Tests | Screening) |

NOTES:
*if INR measured which may indicate severe liver injury (possible Hy's Law), must be reported as an SAE

### Example 3: Vancomycin and Familial Fecal Microbiota Transplant for the Treatment of Subjects with Autism Spectrum Disorder

Objectives: Improve the core features of Autism Spectrum Disorder (ASD) (social interaction, communication and behavioral problems) as well as the short and long-term effects of the outcomes; Improve other non-core aspects of behavior or function such as self-injurious behavior; Improve the quality of life of subjects and their caregivers; And assess gastrointestinal microbiome relative abundance before and after familial fecal microbiota transplant using whole genome shotgun sequencing.

Procedure: This is an open-label clinical trial to evaluate the benefits of familial fecal microbiota transplant following a 6-week treatment with Vancomycin in minor and adult subjects with ASD for treatment of social deficits and language delays.

Participants in the study are required to meet the clinician on 15 occasions, two appointments for baseline assessment, one appointment for the fecal microbiota transplant procedure by colonoscopy, and twelve appointments to for the post-transplant assessment.

Donor Screening: Once the patient has been deemed eligible and baseline measurements have been collected, a suitable donor will be determined. This donor should be a first-degree relative of the patient (parent, sibling, or child). This donor will present to the clinic for vital signs, physical exam, blood draw for laboratory analysis, and will provide a fresh stool sample for testing. Blood tests will include the following: CBC, complete metabolic profile, CMV IgG, EBV Ab panel, Entamoeba histolytica Ab, Hepatitis A Ab, Hepatitis B core Ab, Hepatitis B surface Ab, Hepatitis C Ab, HHV-6 IgG, HIV antibody, HSV 1 & 2 IgG, HTLV-I/II Ab, IgE, Immunoglobulins panel QT IgM, IgG, IgA, JC virus Ab, Lymphocyte subset panel I, Strongyloides stercoralis, and Syphilis serology. Stool tests will include: CRE, ESBLs, GI panel by PCR, H. pylori, and VRE.

Processing of Sample/Dosing: We will treat minors and adults with ASD using vancomycine followed by familial fecal microbiota transplant. The vancomycin will be prescribed at the second baseline visit and will be given for six weeks. This is an open-label study, so blinding is not a component. For FFMT the donor will provide a fresh stool sample the morning of the procedure. This will be brought by the donor to the study site for processing. Processing includes emulsification with sterile normal saline at a 1:1 ratio to create a fecal slurry. Following the FFMT by colonoscopy the caregiver will be taught how to prepare and administer the FFMT enemas at home (during the week 4 visit).

Vancomycin Treatment: Patient will be given a course of vancomycin of 6-week duration. The dose will 250 mg of liquid suspension (formulated in a concentration of 500 mg/6 mL) every 8 hours.

Familial Fecal Microbiota Transplantation by Colonoscopy: The patient will be scheduled for familial fecal microbiota transplantation (FFMT) at an outpatient surgical center, to be conducted the day after completion of the Vancomycin treatment. The patient will prepare for the procedure the day prior by drinking a prescribed regimen of bowel cleanse solution and water. The patent will present for the procedure having fasted and prepped as instructed by the investigator. The patient will undergo the FFMT procedure under anesthesia and be driven home afterwards. Under no circumstances is the patient to drive themselves home.

Familial Fecal Microbiota Transplant by Enema: After 4 weeks the patient will begin FFMT enemas at home utilizing fresh stool from the same donor as the first FFMT. The patient will lie in the lateral decubitus position and the enema will be inserted into the anus. The fecal material will be slowly expelled into the patient's rectum. The patient will remain in the lateral decubitus position for approximately 30 minutes before getting up. The patient may then use the restroom.

Post Treatment Assessment: After FFMT, the patient will be called for monthly post treatment follow-up visits. The patient will bring a fresh stool sample for microbiome testing to each of these visits. The following tests will be administered at these monthly visits: ATEC, CARS-II, CFQL and SRS-II. During the 3, 6, 9, and 12-month follow-up visits the patient will have blood drawn for complete blood count and metabolic panel. Vital signs will be taken, adverse events discussed, and concomitant medications will be updated. At the week 4 visit the patient's family will be shown how to prepare and administer the FFMT by enema. During weeks in which the patient does not have a visit, a phone call will be made to check on the patient, and, starting during week 4, to remind the caregiver to administer the FFMT enema at home).

Table 8 documents the treatment protocol.

TABLE 8

| Intervention | | |
|---|---|---|
| Name | Vancomycin | Familial Fecal Microbiota Transplant |
| Dose | Suspension of 83 mg/mL | Emulsification |
| Formulation | (500 mg/6 mL) | |
| Route of | Oral | Delivered to the colon directly by |
| Administration | | colonoscope in the first trasplant, |
| | | and by enema in all subsequent |
| | | transplants |

Statistical Analysis: The statistical evaluation is performed by an outside statistician using SAS® version 9.3 or higher (Statistical Analysis System, SAS Inc., Cary, NC) software package. Descriptive summaries are given by treatment group and/or overall. The number of subjects within each treatment group of the analysis set is given in each table. Categorical variables are summarized with counts (n) and percentages (%), together with the number of non-missing values. Fisher's exact test for categorical variables is used. The number of non-missing values is used as the denominator for the calculation of percentages. Incidence of adverse events is based on the number of subjects in the respective analysis set and treatment group. Descriptive statistics for continuous variables are comprised of the number of non-missing observations (n), mean, standard deviation (SD), median, minimum (Min) and maximum (Max), if not otherwise stated. Student's t-test and Mann-Whitney for continuous parametric and nonparametric variables are used respectively. ANOVA is used for comparing means between individual groups. When applicable, these summaries are provided by visit. In case of premature withdrawal from the trial, efficacy and safety assessments performed at the time point of withdrawal, are summarized, separately to the planned visits. A P-value<0.05 is taken as significant for all analyses.

### Example 4: Treatment of Over 300 Patients with Familial Fecal Microbiota Transplant

Procedure: Over 300 patients were treated with familial fecal microbiota transplant via the methods outlined above. If the fecal donor member was compatible with the patient, treatment of the patient was successful.

Results: Out of over 300 patients treated, two patients had Rheumatoid arthritis that was healed, one patient had Alzheimer's that improved, two patients had Crohn's disease that improved, and two patients had psoriasis that improved.

Having thus described the invention, it should be apparent that numerous structural modifications and adaptations may be resorted to without departing from the scope and fair meaning of the instant invention as set forth herein above and described herein below by the claims.

1. A method of treating an individual with a health condition, the method comprising the steps of:
a) screening the individual to determine a level of severity of the health condition;
b) acquiring a fecal sample from the individual, wherein the step of acquiring the fecal sample comprises use of a stool sample collection kit or colonoscopy;
c) processing the fecal sample from the individual;
d) sequencing the fecal sample from the individual to determine the individuals microbiome;
e) analyzing the sequenced fecal sample, wherein the step of analyzing the sequenced fecal sample comprises one or more of the following:
i) comparing the individual's microbiome to the microbiome of a parent of individual;
ii) comparing the individual's microbiome to the microbiome of a sibling of the individual;
iii) comparing the individual's microbiome to the microbiome of another individual with same health condition; and
iv) comparing the individual's microbiome with a health condition to the microbiome of the individual before they acquired the health condition;
f) administering 250 mg of liquid vancomycin to the individual every 8 hours for 6 consecutive weeks;
g) performing a fecal microbiota transplant; and
h) monitoring the individual.

2. The method of claim 1, wherein the stool sample kit comprises:
a) at least one stool sample collection vial;
b) at least one toilet accessory or seat cover;
c) at least one specimen bag;
d) at least one pair of gloves;
e) an authorization form;
f) a patient information card;
g) a questionnaire; and
h) stool sample collection instructions.

3. The method of claim 1, wherein the step of administering the vancomycin comprises administering 250 mg of liquid vancomycin to the individual every 8 hours for 10 consecutive days.

4. (canceled)

5. The method of claim 1, wherein a stool sample used for the fecal microbiota transplant is donated from one or more of the following: the individual, a genetic family member of the individual, or a third party unrelated to the individual.

6. The method of claim 1, further comprising after step h) administering at least one fecal microbiota transplant to the individual via enema.

7. A method of treating an individual with a health condition, the method comprising the steps of:
a) screening the individual to determine a level of severity of the health condition;
b) acquiring a fecal sample from the individual;
c) processing the fecal sample from the individual;
d) sequencing the fecal sample from the individual to determine the individuals microbiome;
e) analyzing the sequenced fecal sample;
f) administering 250 mg of liquid vancomycin to the individual every 8 hours for 6 consecutive weeks;
g) performing a fecal microbiota transplant; and
h) monitoring the individual.

8. The method of claim 7, wherein the step of acquiring the fecal sample comprises use of a stool sample collection kit or colonoscopy.

9. The method of claim 8, wherein the stool sample kit comprises:
a) at least one stool sample collection vial;
b) at least one toilet accessory or seat cover;
c) at least one specimen bag;
d) at least one pair of gloves;
e) an authorization form;
f) a patient information card;
g) a questionnaire; and
h) stool sample collection instructions.

10. The method of claim 7, wherein the step of analyzing the sequenced fecal sample comprises one or more of the following:
a) comparing the individual's microbiome to the microbiome of a parent of individual;
b) comparing the individual's microbiome to the microbiome of a sibling of the individual;
c) comparing the individual's microbiome to the microbiome of another individual with same health condition; and
d) comparing the individual's microbiome with a health condition to the microbiome of the individual before they acquired the health condition.

11. The method of claim 7, wherein the step of administering the vancomycin comprises administering 250 mg of liquid vancomycin to the individual every 8 hours for 10 consecutive days.

12. (canceled)

13. The method of claim 7, wherein a stool sample used for the fecal microbiota transplant is donated from one or more of the following: the individual, a genetic family member of the individual, or a third party unrelated to the individual.

14. A method of treating an individual with a health condition, the method comprising the steps of:
a) administering 250 mg of liquid vancomycin to the individual every 8 hours for 6 consecutive weeks; and
b) performing a fecal microbiota transplant, wherein a stool sample used for the fecal microbiota transplant is donated from one or more of the following: the individual, a genetic family member of the individual, or a third party unrelated to the individual.

15. The method of claim 14, wherein the step of administering the vancomycin comprises administering 250 mg of liquid vancomycin to the individual every 8 hours for 10 consecutive days.

16. (canceled)

17. The method of claim 1, wherein the step of analyzing the sequenced fecal sample comprises comparing the individual's microbiome to the microbiome of a parent of individual.

18. The method of claim 1, wherein the step of analyzing the sequenced fecal sample comprises comparing the individual's microbiome to the microbiome of a sibling of the individual.

19. The method of claim 7, wherein the step of analyzing the sequenced fecal sample comprises comparing the individual's microbiome to the microbiome of a parent of individual.

20. The method of claim 7, wherein the step of analyzing the sequenced fecal sample comprises comparing the individual's microbiome to the microbiome of a sibling of the individual.

21. A method of processing and storing fecal samples comprising the steps of:
a) acquiring a fecal sample from the individual;
b) processing the fecal sample from the individual;
c) sequencing the fecal sample from the individual to determine the individuals microbiome;
d) analyzing the sequenced fecal sample, wherein the step of analyzing the sequenced fecal sample comprises one or more of the following:
i) comparing the individual's microbiome to the microbiome of a parent of individual;
ii) comparing the individual's microbiome to the microbiome of a sibling of the individual;
iii) comparing the individual's microbiome to the microbiome of another individual with same health condition; and
iv) comparing the individual's microbiome with a health condition to the microbiome of the individual before they acquired the health condition;
e) freezing and storing the analyzed fecal sample at minus 80 degrees F.

22. The method of claim 21, wherein the fecal sample provided by the individual serves as baseline sample which can then be compared to future fecal samples taken from the same individual to determine a health condition of the individual.

23. The method of claim 21, wherein the frozen fecal sample is thawed and administered to the individual via a fecal transplant.
